(19) United States
(12) Patent Application Publication
(10) Pub. No.: US 2012/0046556 A1
(43) Pub. Date: Feb. 23, 2012

(54) Methods and Kits for Preventing the Spread of Sexually Transmitted Microorganisms

(76) Inventor: Robert M. Block, Leesburg, VA (US)

(21) Appl. No.: 13/091,596

(22) Filed: Apr. 21, 2011

Related U.S. Application Data
(63) Continuation-in-part of application No. 13/084,210, filed on Apr. 11, 2011.
(60) Provisional application No. 61/322,538, filed on Apr. 9, 2010.
(60) Provisional application No. 61/326,393, filed on Apr. 21, 2010.

(51) Int. Cl. A61B 6/00; A61K 31/155; A61P 31/12; A61P 31/22; A61P 31/18; A61M 31/00; A61P 31/10; A61M 3/02; A61M 5/178; A61M 35/00; A61F 13/20; A61K 33/14; A61P 31/04

(52) U.S. Cl. 600/477; 424/662; 514/635; 604/257; 604/187; 604/289; 604/385.17

(57) ABSTRACT

This application is directed to methods and kits for preventing the spread of sexually transmitted microorganisms among individuals. The present method is for the application of a medicament comprising an irrigant to an area of the body having, or which may have, a lesion resulting from, for example, a viral, bacteria or fungal infection, thereby damaging the microorganism within the lesion and preventing the organism from infecting human cells.

### INCORPORATION BY REFERENCE

The present application is a continuation-in part of U.S. application Ser. No. 13/084,210 file on Apr. 11, 2011, which claims priority from Provisional U.S. Patent Application No. 61/322,538 filed on Apr. 9, 2010, the disclosure of which is incorporated herein by reference in its entirety. The present application also claims priority from Provisional U.S. Patent Application No. 61/326,393, filed on Apr. 21, 2010, the disclosure of which is incorporated herein by reference in its entirety.

All documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This application relates to methods and kits for preventing the spread of sexually transmitted microorganisms among individuals. The present method is for the application of a medicament comprising an irrigant to an area of the body having, or which may have, a lesion resulting from, for example, a viral, bacteria or fungal infection, thereby damaging the microorganism within the lesion and preventing the spread of the microorganism from one individual to another individual.

### BACKGROUND OF THE INVENTION

### Viral Infection

### Human Papillomavirus

HPV is the most common sexually transmitted infection and may lead to cervical cancer and to genital warts in both males and females. HPV is also associated with rare cases of cancer of the penis. Particularly in gay men, HPV is linked to anal cancers. There are more than 100 HPV types that can infect the genital areas of males and females, and these HPV types can also infect the mouth and throat. Most people who become infected with HPV do not even know they have it.

HPV is passed on through genital contact, most often during vaginal and anal sex; however, HPV may also be passed on during oral sex and genital-to-genital contact. HPV can be passed on between straight and same-sex partners—even when the infected partner has no signs or symptoms.

A person can have HPV even if years have passed since he or she had sexual contact with an infected person. Most infected persons do not realize they are infected or that they are passing the virus on to a sex partner. It is also possible to contract more than one type of HPV.

At present there are several ways that people can lower their chances of getting HPV, including abstinence, use of condoms and vaccination.

For those who choose to be sexually active, condoms may lower the risk of HPV infection, but HPV can infect areas that are not covered by a condom—so condoms may not fully protect against HPV.

Vaccines can protect males and females against some of the most common types of HPV. Two vaccines (Gardasil® and Cervarix®) are available to protect females against the types of HPV that cause most cervical cancers. One of these vaccines, Gardasil®, also protects both females and males against most genital warts. It targets four strains of human papillomavirus (HPV)—HPV-6, 11, 16, and 18. HPV-16 and HPV-18 account for about 70% of all cervical cancers, and HPV-6 and -11 cause about 90% of genital warts. Cervarix® targets two HPV strains, HPV-16 and HPV-18. However, if a female is already infected with HPV, the vaccine will not prevent that strain of HPV from causing disease. And while vaccination may protect against new infections with other strains of HPV included in the vaccine, there are still more than 100 HPV types for which a vaccine is not available.

Cervical cancer is the second most common cancer in women worldwide. There are about 500,000 new cases, and 250,000 cervical-cancer deaths each year. According to the World Health Organization, nearly 80% of cases occur in low-income countries, where cervical cancer is the most common cancer in women.

People can also lower their chances of getting HPV by being in a faithful relationship with one partner; limiting their number of sex partners; and choosing a partner who has had no or few prior sex partners. Though, even people with only one lifetime sex partner can get HPV, and it may not he possible to determine if a partner who has been sexually active in the past is currently infected.

At present, there is no way to prevent the transmission of HPV between individuals who are not vaccinated or to prevent the transmission of HPV strains for which there is no vaccination.

### Herpes Virus

Herpes simplex virus 1 and 2 (HSV-1 and HSV-2), also known as Human herpes virus 1 and 2 (HHV-1 and -2), are two members of the herpes virus family, Herpesviridae, that infect humans [Ryan K J, Ray C G (editors) (2004). Sherris Medical Microbiology (4th ed.). McGraw Hill. pp. 555-62]. Both HSV-1 and -2 are ubiquitous and contagious. They can be spread when an infected person is producing and shedding the virus.

Symptoms of herpes simplex virus infection include watery blisters in the skin or mucous membranes of the mouth, lips or genitals [Ryan and Ray (2004)]. Lesions heal with a scab characteristic of herpetic disease. However, as neurotropic and neuroinvasive viruses, HSV-1 and -2 persist in the body for the life of the carrier by becoming latent and hiding from the immune system in the cell bodies of nerves. After the initial or primary infection, some infected people experience sporadic episodes of viral reactivation or outbreaks. In an outbreak, the virus in a nerve cell becomes active and is transported via the nerves axon to the skin, where virus replication and shedding occur and cause new sores [“Herpes simplex”. DemNet NZ—New Zealand Dermatological Society. (2006) Retrieved 2006 Oct. 15].

There is no known cure for HSV infection, but treatments can reduce the likelihood of viral shedding and spread.

### Human Immunodeficiency Virus

Human immunodeficiency virus (HIV) is a lentivirus (a member of the retrovirus family) that causes acquired immunodeficiency syndrome (AIDS) [Weiss R A (May 1993). “How does HIV cause AIDS?” Science 260 (5112): 1273-9; Douek D C, Roederer M, Koup R A (2009) “Emerging concepts in the immunopathogenesis of AIDS”. Annu. Rev. Med. 60: 471-84], a condition in humans in which the immune system begins to fail, leading to life-threatening opportunistic infections. Infection with HIV occurs by the transfer of blood, semen, vaginal fluid, pre-ejaculate, or breast milk. Within these bodily fluids, HIV is present as both free virus particles and virus within infected immune cells. The four major routes of transmission are unsafe sex, contaminated needles, breast milk, and transmission from an infected mother to her baby at birth (vertical transmission). Screening of blood products for HIV has largely eliminated transmission through blood transfusions or infected blood products in the developed world.

HIV infection in humans is considered pandemic by the World Health Organization (WHO). From its discovery in 1981 to 2006, AIDS killed more than 25 million people [Joint United Nations Programme on HIV/AIDS (2006). “Overview of the global AIDS epidemic” (PDF). 2006 Report on the global AIDS epidemic. Retrieved 2006 Jun. 8]. HIV infects about 0.6% of the world's population. In 2005 alone, AIDS claimed an estimated 2.4-3.3 million lives, of which more than 570,000 were children. A third of these deaths are occurring in Sub-Saharan Africa, retarding economic growth and increasing poverty [Greener, R. (2002). “AIDS and macroeconomic impact”. in S, Forsyth (ed.). State of The Art: AIDS and Economics. IAEN. pp. 49-55]. According to current estimates, HIV is set to infect 90 million people in Africa, resulting in a minimum estimate of 18 million orphans [Joint United Nations Programme on HIV/AIDS. “AIDS epidemic update, 2005” (PDF). Retrieved 2006 Feb. 28]. Antiretroviral treatment reduces both the mortality and the morbidity of HIV infection, but routine access to antiretroviral medication is not available in all countries [Palella et al. (1998). “Declining morbidity and mortality among patients with advanced human immunodeficiency virus infection. HIV Outpatient Study Investigators”. M Engl. J. Med 338 (13): 853-860].

Most untreated people infected with HIV-1 eventually develop AIDS. These individuals mostly die from opportunistic infections or malignancies associated with the progressive failure of the immune system [Lawn S D (2004), “AIDS in Africa; the impact of coinfections on the pathogenesis of HIV-1 infection”. J. Infect. Dis. 48 (1): 1-12]. HIV progresses to AIDS at a variable rate affected by viral, host, and environmental factors; most will progress to AIDS within ten years of HIV infection: some will have progressed much sooner, and sonic will take much longer [Buchbinder et al (1994). “Long-term HIV-1 infection without immunologic progression”, AIDS 8 (8): 1123-8; Time from HIV-1 seroconversion to AIDS and death before widespread use of highly-active antiretroviral therapy: a collaborative re-analysis. Collaborative Group on AIDS Incubation and HIV Survival including the CASCADE EU Concerted Action. Concerted Action on SeroConversion to AIDS and Death in Europe”. Lancet 355 (9210): 1131-7. April 2000]. Treatment with anti-retrovirals increases the life expectancy of people infected with HIV. Even after HIV has progressed to diagnosable AIDS, the average survival time with antiretroviral therapy was estimated to be more than 5 years as of 2005 [Schneider et al, (2005). “Patterns of the hazard of death after AIDS through the evolution of antiretroviral therapy, 1984-2004”. AIDS 19 (17): 2009-18.]. Without antiretroviral therapy, someone who has AIDS typically dies within a year [Morgan et al. (2002). “HIV-1 infection in rural Africa: is there a difference in median time to AIDS and survival compared with that in industrialized countries?”. AIDS 16 (4): 597-632.].

### Bacterial Infection

### Bacterial Vaginosis

Bacterial vaginosis (BV) is the most common cause of vaginal infection. It is less commonly referred to as vaginal bacteriosis [“Vaginal Infections—How to Diagnose and Treat Them: Bacterial Vaginosis or Vaginal Bacteriosis”. Medscape. Retrieved 10 Oct. 2009]. It is not necessarily considered to be a sexually transmitted infection but often develops after intercourse with a new partner and is more common in women with multiple partners. BV is caused by an imbalance of naturally occurring bacterial flora, and should not be confused with yeast infection (candidiasis), or infection with Trichomonas vaginalis (trichomoniasis) which are not caused by bacteria.

### Chancroid

Chancroid (also known as “Soft chancre” [James et al. (2006). Andrews' Diseases of the Skin: clinical Dermatology. Saunders Elsevier] and “Ulcus molle” [Rapini et al. (2007), Dermatology: 2-Volume Set, St. Louis: Mosby]) is a sexually transmitted infection characterized by painful sores on the genitalia. Chancroid is known to be spread from one to another individual through sexual contact.

Chancroid is a bacterial infection caused by the fastidious Gram-negative streptobacillus Haemophilus ducreyi. It is a disease found primarily in developing countries, associated with commercial sex workers and their clientele.

Infection levels are low in the western world, typically around one case per two million of the population (Canada, France, UK. and USA). Most individuals diagnosed with chancroid have visited countries or areas where the disease is known to occur frequently, although outbreaks have been observed in association with crack cocaine use and prostitution.

Chancroid is a risk factor for contracting HIV, due to their ecological association or shared risk of exposure, and biologically facilitated transmission of one infection by the other.

### Chlamydia

Chlamydia infection is a common sexually transmitted infection in humans caused by the bacterium Chlamydia trachomatis. The term Chlamydia infection can also refer to infection caused by any species belonging to the bacteria/family Chlamydiaceae. C. trachomatis is found only in humans. Chlamydia is a major infectious cause of human genital and eye disease. Chlamydia infection is one of the most common sexually transmitted infections worldwide; it is estimated that about 1 million individuals in the United States are infected with Chlamydia [Chlamydia fact sheet from the Centers for Disease Control and Prevention].

C. trachomatis naturally found living only inside human cells. Chlamydia can be transmitted during vaginal, anal, or oral sex, and can be passed from an infected mother to her baby during vaginal childbirth. Between half and three-quarters of all women who have a chlamydia infection of the neck of the womb (cervicitis) have no symptoms and do not know that they are infected.

In men, infection of the urethra (urethritis) is usually symptomatic, causing a white discharge from the penis with or without pain on urinating (dysuria). Occasionally, the conditions spreads to the upper genital tract in women (causing pelvic inflammatory disease) or to the epididymis in men (causing epididymitis). If untreated, chlamydial infections can cause serious reproductive and other health problems with both short-term and long-term consequences.

Chlamydial infection of the neck of the womb (cervicitis) is a sexually transmitted infection which is asymptomatic for about 50-70% of women infected with the disease. The infection can be passed through vaginal, anal, or oral sex. Of those who have an asymptomatic infection that is not detected by their doctor, approximately half will develop pelvic inflammatory disease (PID), a generic term for infection of the uterus, fallopian tubes, and/or ovaries. PID can cause scarring inside the reproductive organs, which can later cause serious complications, including chronic pelvic pain, difficulty becoming pregnant, ectopic (tubal) pregnancy, and other dangerous complications of pregnancy.

Chlamydia is known as the “Silent Epidemic” because in women, it may not cause any symptoms in 75% of cases [“FreeTest.Me—About Chlamydia”. Retrieved 2008 Dec. 15], and can linger for months or years before being discovered. Symptoms that may occur include: unusual vaginal bleeding or discharge, pain in the abdomen, painful sexual intercourse (dyspareunia), fever, painful urination or the urge to urinate more frequently than usual (urinary urgency).

In men, Chlamydia shows symptoms of infectious urethritis (inflammation of the urethra) in about 50% of cases [“FreeTest.Me—About Chlamydia”. Retrieved 2008 Dec. 15].

Symptoms that may occur include: a painful or burning sensation when urinating, an unusual discharge from the penis, swollen or tender testicles, or fever. Discharge, or the purulent exudate, is generally less viscous and lighter in color than for gonorrhea. If left untreated, it is possible for Chlamydia in men to spread to the testicles causing epididymitis, which in rare cases can cause sterility if not treated within 6 to 8 weeks. Chlamydia is also a potential cause of prostatitis in men, although the exact relevance in prostatitis is difficult to ascertain due to possible contamination from urethritis [Wagenlehner et al. (2006). “Chlamydial infections and prostatitis in men”. BJU Int. 97 (4): 687-90].

### Grannuloma Inguinale

Granuloma inguinale is a bacterial disease that has readied endemic proportions in many underdeveloped regions. Because of the scarcity of medical treatment, the disease often goes untreated. The disease is characterized by painless genital ulcers which. can be mistaken fur syphilis [Murray et al. (2005), Medical Microbiology, fifth ed., Elsevier Mosby, p. 336.]. However, they ultimately progress to destruction of internal and external tissue, with extensive leakage of mucus and blood from the highly vascular “beefy red” lesions.

The microorganism spreads from one host to another through contact with the open sores.

### Gonorrhea

Gonorrhea (also gonorrhoea) is a common sexually transmitted infection caused by the bacterium Neisseria gonorrhoeae. In the US, its incidence is second [CDC—STD Surveillance—Gonorrhea”. Retrieved 2008 Aug. 21 ] only to chlamydia among bacterial STDs [CDC Fact Sheet—Chlamydia”,Retrieved 2008 Aug. 21]. In both men and women if gonorrhea is left untreated, it may spread throughout the body, affecting joints and even heart valves.

The infection is transmitted from one person to another through vaginal, oral, or anal sexual relations, though transmission occurs rarely with safe sex practices of condom usage. The incubation period is 2 to 30 days with most symptoms occurring between 4-6 days after being infected. A small number of people may be asymptomatic for a lifetime. Between 30% and 60% of people with gonorrhea are asymptomatic or have subclinical disease [Y T van Duynhoven (1999). “The epidemiology of Neisseria gonorrheae in Europe”, Microbes and Infection 1 (6): 455-464].

### Syphilis

Syphilis is a sexually transmitted disease caused by the spirochetal bacterium Treponema pallidum subspecies pallidum. The route of transmission of syphilis is almost always through sexual contact, although there are examples of congenital syphilis via transmission from mother to child in utero.

The signs and symptoms of syphilis are numerous; before the advent of serological testing, precise diagnosis was very difficult. In fact, the disease was dubbed the “Great Imitator” because it was often confused with other diseases, particularly in its tertiary stage.

Syphilis can generally be treated with antibiotics, including penicillin. If left untreated, syphilis can damage the heart, aorta, brain, eyes, and bones. In some cases these effects can be fatal.

Primary syphilis is typically acquired via direct sexual contact with the infectious lesions of a person with syphilis [Pickering L K, ed (2006). “Syphilis”, Red Book, Elk Grove Village, Ill.: American Academy of Pediatrics. pp. 631-644]. Approximately 10-90 days after the initial exposure (average 21 days) a skin lesion appears at the point of contact, which is usually the genitalia, but can be anywhere on the body. This lesion, called a chancre, is a firm, painless skin ulceration localized at the point of initial exposure to the spirochete, often on the penis, vagina or rectum. In rare circumstances, there may be multiple lesions present, although it is typical that only one lesion is seen.

### Trichomoniasis

Trichomoniasis, sometimes referred to as “trich”, is a common cause of vaginitis. It is a sexually transmitted disease. It is caused by the single-celled protozoan parasite Trichomonas vaginalis. Trichomoniasis is primarily an infection of the urogenital tract; the most common site of infection is the urethra and the vagina in women.

The American Social Health Association estimates trichomoniasis affects 7.4 million previously unaffected Americans each year and is the most frequently presenting new infection of the common sexually transmitted diseases [Associated Press, Abstinence students still having sex, MSNBC, Apr. 16, 2007. Retrieved Mar. 12, 2008].

Use of male condoms may help prevent the spread of trichomoniasis [Vaginitis/Trichomoniasis: Reduce your risk, American Social Health Association. Retrieved Mar. 12, 2008], although careful studies have never been done that focus on how to prevent this infection.

### Fungal Infection

### Tinea Cruris “Jock Itch”

As the common name for this condition implies, it causes itching or a burning sensation in the groin area, thigh skin folds, or anus. It may involve the inner thighs and genital areas, as well as extending back to the perineum and perianal areas. Affected areas may appear red, tan, or brown, with flaking, rippling, peeling, or cracking skin.

The acute infection begins with an area in the groin fold about a half-inch. across, usually on both sides. The area may enlarge, and other sores may develop in no particular pattern. The rash appears as raised red plaques (platelike areas) and scaly patches with sharply defined borders that may blister and ooze.

If the rash advances, it usually advances down the inner thigh. The advancing edge is redder and more raised than areas that have been infected longer. The advancing edge is usually scaly, and very easily distinguished or well demarcated. The skin within the border turns a reddish-brown and loses much of its scale. The border may exhibit tiny pimples or even pustules, with central areas that are reddish and dry with small scales. If infected with candidal organisms, the rash tends to be redder and wetter. The skin of the penis may be involved, whereas other organisms spare the penis.

Opportunistic infections (infections that are caused by a diminished immune system) are frequent. Fungus from other parts of the body (commonly tinea pedis or ‘athlete's foot’) can contribute to jock itch. A warm, damp environment allowing the fungus to cultivate greatly contributes; especially with tight, sweaty or rubbing clothing such as a jockstrap.

The type of fungus that most commonly causes tinea cruris is called Trichophyton rubrum. Some other contributing fungi are Candida albicans, Trichophyton mentagrophytes and Epidermophyton floccosum.

### Candidiasis “Yeast Infection”

Candidiasis or thrush is a fungal infection (mycosis) of any of the Candida species, of which Candida albicans is the most common. Candidiasis encompasses infections that range from superficial, such as oral thrush and vaginitis, to systemic and potentially life-threatening diseases. Candida infections of the latter category are also referred to as candidemia and are usually confined to severely immunocompromised persons, such as cancer, transplant, and AIDS patients.

Superficial infections of skin and mucosal membranes by Candida causing local inflammation and discomfort are however common in many human populations [MedlinePlus Encyclopedia Vaginal yeast infection]. While clearly attributable to the presence of the opportunistic pathogens of the genus Candida, candidiasis describes a number of different disease syndromes that often differ in their causes and outcomes [Fidel P L (2002), “Immunity to Candida”. Oral Dis. 8: 69-75]. Commonly referred to as a yeast infection, it is also technically known as candidosis, moniliasis, and oidiomycosis [James, William D.; Berger, Timothy G.; et al. (2006) Andrews' Diseases of the Skin: clinical Dermatology. Saunders Elsevier. ISBN 0-7216-2921-0].

Candida yeasts are commonly present in humans, and their growth is normally limited by the human immune system and by other microorganisms, such as bacteria occupying the same locations (niches) in the human body [Mulley, A. G.; Goroll, A. H. (2006). Primary Care Medicine: office evaluation and management of the adult patient. Philadelphia: Wolters Kluwer Health. pp. 802-3].

A weakened or undeveloped immune system or metabolic illnesses such as diabetes are significant predisposing factors of candidiasis [Odds F C (1987), “Candida infections: an overview”. Crit. Rev. Microbiol. 15 (1): 1-5] Diseases or conditions linked to candidiasis include HIV/AIDS, mononucleosis, cancer treatments, steroids, stress, and nutrient deficiency. Almost 15% of people with weakened immune systems develop a systemic illness caused by Candida species. In extreme cases, these superficial infections of the skin or mucous membranes may enter into the bloodstream and cause systemic Candida infections.

In penile candidiasis, the causes include sexual intercourse with an infected individual, low immunity, antibiotics, and diabetes. Male genital yeast infection is less common, and incidence of infection is only a fraction of that in women; however, yeast infection on the penis from direct contact via sexual intercourse with an infected partner is not uncommon [David L M, Walzman M, Rajamanoharan S (October 1997). “Genital colonisation and infection with candida in heterosexual and homosexual males”. Genitourin Med 73 (5): 394-6].

### Antimicrobial Agents

One way to prevent the transmission of a microorganism among individuals is to damage or disrupt the organism cell wall thereby preventing the organism from binding to and infecting human cells. This may be accomplished by applying an antimicrobial (e.g., antibacterial, antiviral) agent to the microorganism.

### Botanical and Herbal Antimicrobials

Berberine and berbine, found in golden seal, barberry and Oregon grape, are the alkaloids responsible for the antibiotic actions of many botanicals. Berberine has been shown to increase the blood supply to the spleen and to promote the release of compounds such as tuftsin that potentiate immune function. Berberine has also been shown to activate macrophages.

Berberine has shown antimicrobial activity against bacteria, protozoa, and fungi, including: Staph sp., Strep. sp., Chlamydia sp., Corynebacterium diphtheria, E. coli, Salmonella typhi, Vibrio cholerae, Diplococcus pneumonia, Pseudomonas sp., Shigella dysenteriae, Entamoeba histolytica, Trichomonas vaginalis, Neisseria gonorrhoeae and meningitis, Treponema pallidum, Giardia lamblia, and Leishmania donovani. Berberine's action against Candida may be stronger than that of antibiotics commonly used for these pathogens. Similarly, berbine exhibits antimicrobial activity.

### Dental Irrigants

For example, endodontic root canal procedures include the use of irrigants such as sodium hypochlorite (bleach), Tubulicid, and chlorhexidine (Peridex) to prevent microorganisms from invading dentinal tubules. Sodium hypochlorite is universally used as an antiseptic for root canal irrigation, its principal functions in root canal treatment being microbicidal and dissolving organic material. Chlorhexidine is an antiseptic agent used as an antibacterial dental rinse, and is essentially nontoxic when applied to the skin or mucous membranes. These reagents are FDA-approved and considered both safe and effective for use in destroying microbes.

### Plasma Gas

Another way to avis the body of microorganisms is to use plasma, which at room temperature and pressure, has been engineered as a disinfectant to kill microorganisms, quickly inactivating not only bacteria but also viruses and fungi [Eisenberg, Anne. “Hospital-Clean Hands, Without All the Scrubbing”, NY Times, Published Feb. 13, 2010].

There have been many documented cases of plasmas being applied for sanitizing skin or other body parts. Plasma cleaners make their antibacterial cocktails by running electrical current through air. The electric current ionizes the oxygen, nitrogen and water vapor in the air, eventually creating nitric oxide, hydrogen peroxide and particles effective against bacteria.

Citation or identification of any document in this application is not an admission that such document is available as prior art to the present invention.

### SUMMARY OF THE INVENTION

The present application relates to methods and kits for preventing the spread of sexually transmitted microorganisms, including viruses, bacteria and fungi. The present invention is directed toward methods and kits comprising a medicament comprising an irrigation solution, for example, a dental irrigation solution, for preventing the transmission of sexually transmitted microorganisms. The medicament may be used alone or in combination with an antimicrobial ionized alkaline wash and/or plasma luminous gas.

Thus, it is an object of this invention to provide a method of preventing the transmission of microorganisms, comprising identifying an individual having a sexually transmitted microorganism and applying to a body orifice or genitalia of an individual a medicament comprising a therapeutically effective amount of an irrigant.

It is also an object of this invention to provide a method of identifying in an individual a lesion caused by a microorganism, said method comprising: (a) applying to a body orifice or genitalia a medicament comprising an irrigant and an ultraviolet-induced fluorescent dye, wherein the fluorescent dye may hind to the viral or bacterial cell wall; and (b) exposing the body orifice or genitalia to ultraviolet light such that the light may react with the fluorescent dye, wherein the cell wall of the virus or bacteria is highlighted, thereby identifying a viral or bacterial lesion.

In one embodiment of the invention, the medicament may further comprise a pharmaceutically acceptable carrier or excipient.

In one embodiment of the invention, the microorganism may be a sexually transmitted virus, bacterium or fungus. The microorganism may be, but is not limited to, human papillomavirus, herpes virus, human immunodeficiency virus, bacterial vaginosis, chancroid, chlamydia, grannuloma inguinale, gonorrhea, syphilis, trichomoniasis, tinea cruris, or candidiasis. In one embodiment, the sexually transmitted virus is human papillomavirus.

In one embodiment of the invention, the irrigant may include, but is not limited to a hypochlorite compound, chlorohexidine compound, quaternary ammonium compounds, parachlorometaxylenol compounds, gluteraldehyde compounds, phenolic compounds, peroxyacetic acid, alcohols, chlorine dioxide, chloroxyenols, tetracyclines, iodine, cresols, caprylic acid, formaldehyde, and trichlorosan compounds. In one embodiment, the irrigant comprises sodium hypochlorite chlorohexidine. The irrigants of the present invention may be present in the medicament in an amount between about 0.1% and about 20% w/w (weight by weight) of the medicament. In another embodiment, the irrigant is present in an amount between about 0.1% and about 5.0% w/w of the medicament.

In one embodiment of the invention, the medicament may have a pH between about 2.0 and about 11.0, although the pH may be lower or higher, depending on the choice of irrigant. In one embodiment, the medicament may have a pH between about 5.0 and about 7.0.

The term “about” as used herein, refers to an approximate 10% variation.

In one embodiment of the invention, the medicament may further comprise one or more antimicrobial agent and/or an antiseptic agent. A antimicrobial agent may be an antibacterial agent or an antiviral agent Antimicrobial agents of the present invention include, but are not limited to, penicillins, cephalosporins, cephamycins, carbopenems, monobactam, vancomycin, teicoplanin, macrolides, tetracyclines, aminoglycosides, chloramphenicol, sodium fusidate, sulphonamides, quinolones, azoles, and Noni (morinda citrifolia, a citric acid fruit juiced used in Polynesia as an antibacterial, antiviral and antifungal agent). Antimicrobial agents of the present invention may also include botanical and herbal extracts, including but not limited to, goldenseal or grape extract. In one embodiment, the botanical or herbal extract may comprise berberine alkaloid (berberine) or berbine. In one embodiment, the antimicrobial agent may be present in an amount of between about 0.1% and about 20% w/w of the medicament. In another embodiment, the antimicrobial agent is present in an amount of between about 0.1% and about 5.0% w/w of the medicament.

Antiseptic agents used in the present invention include, but are not limited to benzalkonium chloride, cetrimide, hexachlorophene, iodine compounds, mercury compounds, alcohol, hydrogen peroxide, hexamine hippurate, triclosan, cetylpyridinium chloride, and dequalinium, boric acid, volatile oils, and botanical essential oils. In one embodiment of the invention, the antiseptic agent may be present in an amount of between about 0.1% and 20% w/w of the medicament. In another embodiment of the invention, the antiseptic agent is present in an amount of between about 0.1% and 5.0% w/w of the medicament.

It is to be understood that the irrigants of the present invention may comprise antimicrobial agents and/or antiseptic agents. In one embodiment the antimicrobial agent may he an antiseptic agent. In another embodiment, the antimicrobial agent and/or antiseptic agent may be the irrigant comprised within the medicament.

In one embodiment of the invention, the medicament may further comprise one or more surfactant. Surfactants of the present invention may include, but are not limited to, fluorosurfactants, ethoxylates, sulfonates, quaternary ammonium compounds, and amine oxides. In one embodiment, the surfactant may be present in an amount of between about 0.1% and 5% w/w of the medicament.

In one embodiment of the invention, the body orifice or genitalia includes, but is not limited to a vagina, a penis, a rectum and a mouth.

In one embodiment of the invention, the medicament may be in the form of a solution, a gel, a water-in-oil emulsion or a suspension.

In one embodiment of the invention, the medicament may be administered using an applicator. An applicator of the present invention may be, but is not limited to, a wipe, a measuring cup, a douche, an enema, a syringe, a tampon and a spray.

In one embodiment of the invention, the medicament may further comprise a taste-masking agent. In another embodiment, the taste-masking agent may be a non-nutritive sweetener. In one embodiment, the medicament is in the form of an oral rinse comprising a taste-masking agent.

In one embodiment of the invention, the medicament may further comprise a fluorescent dye that may bind to a cell wall of the virus or bacteria. The fluorescent dye may be an ultraviolet-induced fluorescent dye.

In one embodiment of the invention, the method may further comprise exposing the body orifice or genitalia to an ultraviolet light such that the light reacts with the fluorescent dye, wherein the damaged cell wall of the virus or bacteria is highlighted, thereby identifying a viral or bacterial lesion.

In one embodiment of the invention, the medicament may be administered before sexual intercourse. In another embodiment, the medicament may he administered after sexual intercourse. In yet another embodiment, the medicament is administered before and after sexual intercourse.

In one embodiment of the invention, the medicament may be applied for a period of time between about 1 minute and about 5 minutes. In another embodiment, the medicament may be applied to the body orifice multiple, consecutive periods of time.

In one embodiment of the invention, the method may further comprise applying to the body orifice a wash comprising ionized alkaline water, wherein the wash may be applied before and/or after the medicament, and wherein the wash establishes a particular pH in the body orifice.

In one embodiment of the invention, the method may further comprise applying to the body orifice a plasma gas.

In one embodiment of the invention, the medicament may comprise arginine.

It is also an object of this invention to provide a kit fur preventing the transmission of a microorganism, comprising: (a) a medicament comprising an irrigant; (b) an applicator; and (c) optionally, a prophylactic device.

In one embodiment of the invention, the kit may further comprise a wash comprising ionized alkaline water and an applicator for said wash.

In another embodiment of the invention, the kit may further comprise a fluorescent dye that may bind to a damaged cell wall of the virus or bacteria. The fluorescent dye may be an ultraviolet-induced fluorescent dye.

In one embodiment of the invention, the prophylactic device may be, but is not limited to, a condom, spermicide, or a dam.

In one embodiment of the invention, the kit may further comprise a plasma gas.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

As summarized above, the present invention discloses a method of preventing the spread of sexually transmitted microorganisms among individuals. In particular, the invention discloses the use of irrigants, for example, dental irrigants, in a medicament for the purpose of preventing microorganisms from infecting human cells. The epithelial cell structure of the oral cavity is similar to that of the vaginal cavity, and both cavities are subject to viral, bacterial, and fungal lesions.

It is to be understood that all agents of the instant invention are to be used at therapeutically effective concentrations. When referring to an irrigant, an antimicrobial agent, an irrigant comprising one or more antimicrobial agents and the like, a therapeutically effective concentration of an agent may be one which will be effective in damaging a microorganism (e.g., damaging the cell wall of the microorganism) in a lesion residing in a body orifice or on genitalia of an individual, rendering the microorganism unable to infect a human cell.

The therapeutically effective amount of an agent administered according to a method within the scope of the disclosed invention may vary according to age, weight, height, sex, area of target region, number of applications, skin thickness, responsiveness to medicament and other individual variables known. For example, the extent of the area of tissue influenced is believed to be proportional to the volume of medicament applied, while the severity of damage to the microorganism may be, for most dose ranges, proportional to the concentration of agent administered. The amount and concentration of agent may also depend on the solubility characteristics of the chosen agent.

As used herein, “sexually transmitted” refers to the spread or transmission from one individual to another through sexual intercourse or other sexual contact.

As summarized above, this invention discloses the use of a medicament comprising an irrigant, which is designed for use in body orifices and/or on genitalia, The medicament may be in the form of a solution, a gel, a water-in-oil emulsion, or a suspension. This medicament may also comprise one or more antimicrobial agents, including antiviral and antifungal agents. This medicament may also comprise one or more antiseptic agents and surfactants. By using this type of medicament, shortly before and/or shortly after sexual intercourse, the irrigant may reduce the risk of a previously uninfected person becoming infected by sexually transmitted microorganisms, such as human papillomavirus, herpes virus, human immunodeficiency virus, bacterial vaginosis, chancroid, chlamydia, grannuloma inguinale, gonorrhea, syphilis, trichomoniasis, tinea cruris, and candidiasis.

To provide maximal protection, this type of irrigant-containing medicament may be used both before and after each act of sexual contact in which there is a significant risk of transmission of a sexually transmitted microorganism. However, a substantial degree of protection can be provided by a single use, either before or after the sexual encounter. In particular, a recent report (Vernazza 2001) has indicated that HIV particles are actively shed into vaginal fluids, even among women who are taking anti-HIV drugs that suppress viral concentrations in the blood to very low or even undetectable levels.

An irrigant-containing medicament as disclosed herein may be used in conjunction with a prophylactic device, during intercourse in which a significant risk of sexually transmitted infection exists; however, since that doesn't always happen, this type of medicament may be used regardless of whether a prophylactic is also used, to provide at least some increased level of protection.

An irrigant-containing medicament as disclosed herein may be formulated for periodic use, such as a few times each week, or on a daily basis.

As used herein, the term “medicament” may include “genital lubricants”. As used herein, a “genital lubricant” is a friction-reducing formulation that is effective, desirable, and comfortable, when used topically, as a lubricant, during intercourse. As such, a genital lubricant (as that term is used herein) must contain at least one agent that is conventionally used as a lubricating agent. Glycerin (also called glycerine, glycerol, 1,2,3-propanetriol, and trihydroxypropane) and certain types of polyethylene glycol (PEG), such as PEG 200 or PEG 400 (the numbers indicate different molecular weight averages) are widely used as lubricating agents in topical formulations. Various other polymers (such as polypropylene glycol, polyisobutene, and polyoxyethylene) and behenic acid and behenyl alcohol are also used as lubricants in cosmetics and other formulations that contact the skin. In addition, some sugar-alcohols such as sorbitol, and some silicon compounds such as polydimethyl-siloxane, are also used as skin and/or genital lubricants. When rubbed between two fingers, it quickly becomes clear that any of these agents will impart a “slippery” feel to the skin, in a manner which easily surpasses the lubricating traits of water.

As part of its ability to impart sustained lubrication and comfort throughout a complete act of intercourse, an effective genital lubricant must have an affinity for skin. This is evidenced by two distinct but consistent and overlapping factors. First, a lubricant will remain on the skin, as a fairly consistent and uniform coating, for a substantially longer period of time than water, or a rinsing formulation that does not contain a lubricant. Secondly, most types of lubricants, if allowed to dry on the skin without being wiped off, will leave a residue, usually in the form of a film, which often goes through a somewhat adherent “sticky” phase, and which may remain sticky until washed off.

In a separate embodiment, it may he preferable to have a medicament which explicitly excludes lubricating ingredients such as glycerin, polyethylene glycol, etc., will not create the type of coating layer and/or sticky residue that a lubricant will create. The instantly claimed medicament may applied in the form of a rinse and may, instead, behave in a manner which is closer or even directly comparable to the performance of plain water, or salt water, when used to wash off the skin. In the same way that water may be used to rinse off something, a rinse, as defined herein, can also wash things off, and wash things out, in an effective manner that will not leave a sticky residue or film caused by an ingredient in the rinse.

It should be recognized that the medicament of the instant invention may contain a moisturizing ingredient. Moisturizing agents which do not function effectively as lubricants are widely used in, for example, hand and face lotions and cosmetics which are non-oily, and which are designed to generate a clean and non-greasy feeling when used. As just one example, aloe vera gel is widely used as a moisturizing agent, but it would not perform adequately as a lubricating agent, if added to a genital lubricant. Various other such moisturizing ingredients are known to those who manufacture lotions and cosmetics, and may be incorporated if desired into a rinse formulation as disclosed herein, at appropriate concentrations (which in most cases are likely to be in the range of about 0.1. to 5%, by weight).

Medicaments of the present invention may include formulations comprising zinc salts or zinc ions, although in a preferred embodiment, the medicament does not include zinc ions or salts.

Medicaments as disclosed herein may he provided in fully hydrated form, if desired. Alternately, to reduce bulk, shipping costs, and the risk of storage and leakage problems, and to increase their shelf life, they may be sold as powders or concentrates, which may be converted into aqueous or gel-like formulations by dissolving them in water or gel. They may be packaged in any desired manner.

To maximize anti-microbial efficacy when used before intercourse, an irrigant-containing medicament preferably should be used a reasonable and relatively short period of time before intercourse commences, such as within an hour or possibly two hours before intercourse begins. In general, using an irrigant-containing medicament, may have some beneficial effect, if carried out at any time within roughly twelve hours (and possibly more) prior to intercourse. However, since any viral load, for example, in an infected person will tend to return and build up gradually, over a span that may be measured in hours, and since any protective residual irrigant concentrations that remain on accessible genital, rectal or oral surfaces will gradually be absorbed and dissipated over time, a relatively short delay (such as only an hour, or less) before intercourse will provide a better margin of safety and protection than a longer delay (such as more than two hours). Accordingly, as defined herein, “before intercourse” or “prior to intercourse” refers to less than 24 hours before intercourse, preferably less than 12 hours before intercourse, more preferably less than 6 hours before intercourse, and most preferably less than an hour before intercourse.

Since irrigants tend to have an astringent effect, the claimed medicament may be followed by application of a moisturizing and/or lubricating compound (various vaginal moisturizers are well-known in the art). If desired, a moisturizer and/or lubricant containing an antimicrobial agent may be used, for greater antimicrobial efficacy.

To maximize antimicrobial efficacy when used after intercourse, an irrigant-containing medicament should be used within about 10 to 15 minutes after ejaculation. This shorter time limitation is based on data reported in Zacharopoulos et al 1992, Phillips & Bourinbaiar 1992, and Pearce-Pratt & Phillips 1993, which indicate that within about 1.5 minutes, HIV-infected lymphocytes commence a cellular process in which infected cells, rather than free virus particles, begin binding to uninfected epithelial cells, and begin shoving large numbers of HIV particles or genomes at and into the epithelial cells. Accordingly, use of an irrigant containing medicament within about 15 minutes or less after ejaculation is preferable, since it can reduce the risk of that type of cell-to-cell infection process.

However, since the 15 minute period observed in those tests was based on various parameters and conditions (including high-titer viral concentrations) that were designed to facilitate easily observable tests with clear outcomes, it is not believed that a 15 minute delay is a rigid boundary line, beyond which using the instant medicament would he a wasted effort. Instead, it is believed and assumed that post-coital use of the irrigant-containing medicament at any time up to roughly 12 hours after intercourse may be able to significantly reduce the risk of infection. Accordingly, as defined herein, “after intercourse” or “following intercourse” refers to less than 24 hours after intercourse, preferably less than 12 hours after intercourse, more preferably less than 6 hours after intercourse, and most preferably less than an hour after intercourse.

Different formulations and additives may be preferred for pre-coital and post-coital medicaments, since they will remain in contact with the genital membranes for different periods of time. As one example, a pre-coital medicament might contain a moisturizer, to counteract the astringent effects of irrigant, while a post-coital medicament might contain a small concentration of Vitamin E, retinol, and/or other agent(s) that can promote better long-term benefits if allowed to remain in place for a longer period of time.

In general, concentrations of irrigants in medicaments as disclosed herein are likely to range from about 0.1% to about 20% w/w, depending on which particular irrigant is used. Higher concentrations may also be used, if desired. In one approach which takes varying individual factors (such as skin sensitivity, perceived degree of risk, etc) into account, an assortment of different medicaments with varying irrigant concentrations may be sold, and anyone who wishes to use one will be free to several different concentrations, and choose one based on his or her own preference. This is comparable to sunblocking agents being sold with a range of different “sun protection factors”.

Medicaments of the present invention may be used alone or together with a wash comprising alkaline ionized water, or electrolyzed reduced water (ERW). Ionized water is the product of mild electrolysis. It serves to balance the body's pH. A body that is too acidic provides the ideal environment for diseases to manifest and thrive. Thus, an ionized wash used in accordance with the present invention may balance any pH imbalance caused by administration of the claimed irrigant-containing medicaments.

Medicaments of the present invention may also be used with an antimicrobial plasma gas, either administered before or after the medicament is administered.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method of preventing the transmission of microorganisms, comprising:
(a) identifying an individual having a sexually transmitted microorganism; and
(b) applying to a body orifice or genitalia of the individual a medicament comprising a therapeutically effective amount of an irrigant.

2. (canceled)

3. The method of claim 1, wherein the microorganism is a sexually transmitted virus, bacteria, or fungus.

4. The method of claim 3, wherein the microorganism is selected from the group consisting of human papillomavirus, herpes virus, human immunodeficiency virus, bacterial vaginosis, chancroid, chlamydia, grannuloma inguinale, gonorrhea, syphilis, trichomoniasis, tinea cruris, and candidiasis.

5. (canceled)

6. The method of claim 1, wherein the irrigant comprises one or more compounds selected from. the group consisting of: hypochlorite compound, chlorohexidine compound, quaternary ammonium compounds, parachlorometaxylenol compounds, gluteraldehyde compounds, phenolic compounds, peroxyacetic acid, alcohols, chlorine dioxide, chloroxyenols, tetracyclines, iodine, cresols, caprylic acid, formaldehyde, and trichlorosan compounds.

7-10. (canceled)

11. The method of claim 1, wherein the medicament further comprises one or more antimicrobial agent and/or an antiseptic agent.

12-17. (canceled)

18. The method of claim 1, wherein the medicament further comprises one or more surfactant.

19-20. (canceled)

21. The method of claim 1, wherein the body orifice or genitalia is selected from the group consisting of: vagina, penis, rectum and mouth.

22. The method of claim 1, wherein the medicament is in a form selected from the group consisting of: a solution, a gel, a water-in-oil emulsion and a suspension.

23-25. (canceled)

26. The method of claim 1, wherein the medicament further comprises a fluorescent dye that binds to a cell wall of the virus or bacteria.

27-33. (canceled)

34. The method of claim 1, comprising applying to the body orifice or genitalia a wash comprising ionized alkaline water, wherein the wash is applied within 24 hours before the medicament, and wherein the wash establishes a particular pH in the body orifice.

35-37. (canceled)

38. A method-of identifying in an individual a lesion caused by a microorganism, said method comprising:
(a) applying to a body orifice or genitalia a medicament comprising an irrigant and an ultraviolet-induced fluorescent dye, wherein the fluorescent dye binds to the viral or bacterial cell wall; and
(b) exposing the body orifice or genitalia to ultraviolet light such that the light reacts with the fluorescent dye, wherein the cell wall of the virus or bacteria is highlighted, thereby identifying a viral or bacterial lesion.

39. (canceled)

40. The method of claim 38, wherein the microorganism is a sexually transmitted virus, bacteria, or fungus.

41-42. (canceled)

43. The method of claim 38, wherein the irrigant comprises one or more compounds selected. from the group consisting of: hypochlorite compound, chlorohexidine compound, quaternary ammonium compounds, parachlorometaxylenol compounds, gluteraldehyde compounds, phenolic compounds, peroxyacetic acid, alcohols, chlorine dioxide, chloroxyenols, tetracyclines, iodine, cresols, caprylic acid, formaldehyde, and trichlorosan compounds.

44-47. (canceled)

48. The method of claim 38, wherein the medicament further comprises one or more antimicrobial agent and/or an antiseptic agent.

49-64. (canceled)

65. The method of claim 38, comprising applying to the body orifice or genitalia a wash comprising ionized alkaline water, wherein the wash is applied before the medicament, and wherein the wash establishes a particular pH in the body orifice.

66-68. (canceled)

69. A kit for preventing the transmission of a microorganism, comprising:
(a) a medicament comprising an irrigant;
(b) an applicator; and
(c) optionally a prophylactic device.

70. The kit of claim 69, wherein the medicament further comprises a pharmaceutically acceptable carrier or excipient.

71-72. (canceled)

73. The kit of claim 69, wherein the irrigant comprises one or more compounds selected from the group consisting of: hypochlorite compound, chlorohexidine compound, quaternary ammonium compounds, parachlorometaxylenol compounds, gluteraldehyde compounds, phenolic compounds, peroxyacetic acid, alcohols, chlorine dioxide, chloroxyenols, tetracyclines, iodine, cresols, caprylic acid, formaldehyde, and trichlorosan compounds.

74-77. (canceled)

78. The kit of claim 69, wherein the medicament further comprises one or more antimicrobial agent and/or an antiseptic agent.

79-88. (canceled)

89. The kit of claim 69, wherein the applicator is selected from the group consisting of: a wipe, a measuring cup, a douche, an enema, a syringe, a tampon and a spray.

90-97. (canceled)
